(19) United States
(12) Patent Application Publication
(10) Pub. No.: US 2004/0138109 A1
(43) Pub. Date: Jul. 15, 2004

(54) Potent inhibitor of HCV serine protease

(75) Inventors: Shirlynn Chen, Somers, NY (US); Jens Oliver Croenlein, Mittelbiberach (DE); Gerhard Nehmiz, Biberach (DE); Gerhard Steinmann, Erbach-Bach (DE); Jocelyn Abella Gunn, Hamden, CT (US); Phuong Do Costa, Danbury, CT (US)

Correspondence Address: BOEHRINGER INGELHEIM CORPORATION, 900 RIDGEBURY ROAD, P O BOX 368, RIDGEFIELD, CT, 06877, US

(73) Assignees: Boehringer Ingelheim Pharmaceuticals, Inc., Ridgefield, CT (US); Boehringer Ingelheim Pharma GmbH & CO. KG, Ingelheim (DE)

(21) Appl. No.: 10/663,220

(22) Filed: Sep. 16, 2003

Related U.S. Application Data
(60) Provisional application No. 60/414,940, filed on Sep. 30, 2002.
(60) Provisional application No. 60/421,904, filed on Oct. 29, 2002.
(60) Provisional application No. 60/433,834, filed on Dec. 16, 2002.
(60) Provisional application No. 60/443,662, filed on Jan. 30, 2003.

(51) Int. Cl. A61K 38/12; A61K 38/05

(52) U.S. Cl. 514/9; 514/18; 514/312

(57) ABSTRACT

Disclosed are oral pharmaceutical compositions, kits and methods of treating and preventing Hepatitis C Viral (HCV) infections wherein the following Compound (1), a potent inhibitor of HCV serine protease, or a pharmaceutically acceptable salt thereof, is administered in a selected dosage range:

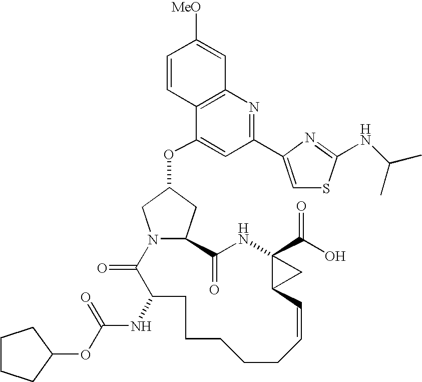

Also disclosed are the use of a compound of formula (1), or a pharmaceutically acceptable salt thereof, as a control substance for validating an HCV replication assay and also as a control substance for determining the relative effectiveness of one or more substances, alone or in combination, to inhibit the replication of HCV.

This application claims the benefit of the following U.S. Provisional Applications: 60/414,940, filed Sep. 30, 2002; 60/421,904, filed Oct. 29, 2002; 60/433,834, filed Dec. 16, 2002; and 60/443,662, filed Jan. 30, 2003; each of which applications are herein incorporated by reference in their entirety.

### FIELD OF THE INVENTION

This invention relates in general to oral pharmaceutical compositions, kits and methods of treating and preventing Hepatitis C Viral (HCV) infections wherein a potent inhibitor of HCV serine protease is used in a selected dosage range.

### BACKGROUND OF THE INVENTION

The inadequate efficacy and tolerability of current therapies for the emerging and devastating infectious liver disease caused by Hepatitis C Virus (HCV) worldwide have warranted significant efforts in the development of new therapeutics. With the insights gained in the design of Human Immunodeficiency Virus (HIV) protease inhibitors for the treatment of AIDS, a similar substrate-based approach was undertaken to design active site inhibitors of the NS3 serine protease with promise in blocking viral replication in HCV-infected patients.

We have reported competitive peptide inhibitors based on N-terminal cleavage products (Llinàs-Brunet et al, Bioorganic & Medicinal Chemistry Letters, 8 (1998), 1713-1718 and 2719-2724). Optimization studies on these peptide inhibitors led to the discovery of the following Compound (1), a small, selective and potent inhibitor of the HCV NS3 serine protease:

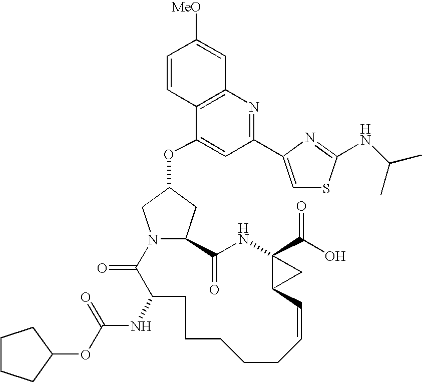

Compound (1) falls within the scope of the macrocyclic peptide series of HCV inhibitors disclosed in WO 00/59929 (Boehringer Ingelheim (Canada) Ltd.) and U.S. application Ser. No. 09/760,946, filed Jan. 16, 2001 (Tsantrizos et al.), which application is herein incorporated by reference. Compound (1) is disclosed as Compound #822 in the aforementioned WO and U.S. application documents.

Compound (1), was selected from an optimized series of inhibitors with potent in vitro activity and adequate pharmacokinetics in various animal species. A distinguishing feature of the Compound (1) inhibitor series is the presence of a C-terminal carboxylic acid functionality. This provides exquisite selectivity with respect to other proteases, a property not easily attained with more conventional classes of covalent, reversible serine protease inhibitors. Inhibitor constant (Ki) values of 0.30 nM and 0.66 nM with a non-covalent, competitive mode of inhibition were obtained for Compound (1) from steady state velocity analysis using the NS3 serine proteases of HCV genotypes 1a and 1b respectively. Compound (1) retains its inhibitory efficacy in human cells and showed low nanomolar inhibition of HCV RNA replication using the replicon cell model system. Mechanism of action studies further demonstrated the ability of Compound (1) to block NS3 protease-dependent polyprotein processing in HCV replicon-containing cells. Compound (1) is orally bioavailable in various animal species. In view of the potent activity in vitro, good PK data in animal models and adequate pre-clinical safety profile, Compound (1) was selected for in-depth clinical evaluation in man as a novel antiviral compound class for the treatment of HCV infection.

In a first single rising dose trial in healthy male subjects the tolerability and pharmacokinetic parameters of Compound (1) were investigated. Compound (1) was found to be well tolerated up to 2000 mg in healthy male subjects. During a recent clinical study (randomized, placebo-controlled, double-blinded, multi-center trial) in patients with chronic HCV infection, it was discovered that Compound (1) administered in an oral pharmaceutical formulation at a selected dosage range was highly effective at reducing the viral load of HCV infected patients. The degree of viral load reduction upon administration of Compound (1) was a significant and unexpected finding, with some patients even experiencing up to a 3 log reduction in viral load within 48 hours after the first administration of Compound (1).

### SUMMARY OF THE INVENTION

In one embodiment, the present invention is directed to oral pharmaceutical compositions comprising Compound (1), or a pharmaceutically acceptable salt thereof, in an amount of about 25 mg to 500 mg dissolved in at least one solvent selected from polyethylene glycol, ethanol, propylene glycol and water, or mixtures thereof, optionally further containing an antioxidant.

The pharmaceutical compositions can further contain one or more additional active agents selected, for example, from antiviral agents, immunomodulatory agents, other inhibitors of HCV NS3 protease, inhibitors of other targets in the HCV life cycle, HIV inhibitors, Hepatitis A Virus (HAV) inhibitors, Hepatitis B Virus (HBV) inhibitors and liver immunoprotective agents.

In another embodiment, the present invention is directed to a kit comprising:

(a) about 25 mg to 500 mg of Compound (1), or a pharmaceutically acceptable salt thereof, and

(b) at least one of the following additional agents: an antiviral agent, an immunomodulatory agent, another inhibitor of HCV NS3 protease, an inhibitor of another target in the HCV life cycle, an HIV inhibitor, an HAV inhibitor or an HBV inhibitor, or a liver immunoprotective agent.

In another embodiment, the present invention is directed to a method of treating or preventing HCV infection in a mammal comprising administering to said mammal about 50 mg to 1000 mg of Compound (1), or a pharmaceutically acceptable salt thereof, per day in single or multiple doses. Such administration can be via an oral pharmaceutical composition, and the composition can also contain one or more additional active agents selected, for example, from antiviral agents, immunomodulatory agents, other inhibitors of HCV NS3 protease, inhibitors of other targets in the HCV life cycle, HIV inhibitors, HAV inhibitors, HBV inhibitors and liver immunoprotective agents. The combination of Compound (1), or a pharmaceutically acceptable salt thereof, and an HIV inhibitor can be used to treat those patients coinfected with HCV and HIV, and the combination of Compound (1), or a pharmaceutically acceptable salt thereof, and an HAV inhibitor can be used to treat those patients coinfected with HCV and HAV, and the combination of Compound (1), or a pharmaceutically acceptable salt thereof, and an HBV inhibitor can be used to treat those patients coinfected with HCV and HBV. The methods of treatment or prevention herein can be performed on HCV genotype 1 variety or non-genotype 1 variety, acute or chronic HCV infection, and in a wide variety of patient population groups as described more fully herein.

In additional embodiments, the methods of the present invention lead to a HCV viral load reduction of 1, 2 or 3 log in the treated patient within 48 hours after the first administration of the Compound of formula (1), or a pharmaceutically acceptable salt thereof, to the patient.

In another embodiment, the present invention is directed to the use of a compound of formula (1), or a pharmaceutically acceptable salt thereof, as a control substance for validating an HCV replication assay and also as a control substance for determining the relative effectiveness of one or more substances, alone or in combination, to inhibit the replication of HCV.

### DETAILED DESCRIPTION OF THE INVENTION

I. Definitions

Terms not specifically defined herein should be given the meanings that would be given to them by one of skill in the art in light of the disclosure and the context. As used throughout the present application, however, unless specified to the contrary, the following terms have the meaning indicated:

The term “about” means within 10%, preferably within 5%, and more preferably within 1% of a given value or range. For example, “about 25 mg” means from 22.5 to 27.5 mg, preferably from 23.75 to 26.25 mg, and more preferably from 24.75 to 25.25 mg. When the term “about” is associated with a range of values, e.g., “about X mg to Y mg”, the term “about” is intended to modify both the lower (X) and upper (Y) values of the recited range. For example, “about 25 mg to 500 mg” is equivalent to “about 25 mg to about 500 mg”.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “pharmaceutically acceptable salt” means a salt of Compound (1) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19, which is hereby incorporated by reference in its entirety.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, hydroiodic acid, sulfuric acid, sulfamic acid, nitric acid, phosphoric acid, and the like, and organic acids such as acetic acid, trichloroacetic acid, trifluoroacetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, heptanoic acid, hexanoic acid, formic acid, fumaric acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, tromethamine, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

The term “antiviral agent” means an agent (compound or biological) that is effective to inhibit the formation and/or replication of a virus in a mammal. This includes agents that interfere with either host or viral mechanisms necessary for the formation and/or replication of a virus in the mammal. Antiviral agents include, for example, ribavirin, amantadine, VX-497 (merimepodib, Vertex Pharmaceuticals), VX-498 (Vertex Pharmaceuticals), viramidine, XTL-001 and XTL-002 (XTL Biopharmaceuticals), JTK-003/002 (Japan Tobacco) and ISIS-14803 (ISIS Pharmaceuticals).

The term “immunomodulatory agent” means those agents (compounds or biologicals) that are effective to enhance or potentiate the immune system response in a mammal. Immunomodulatory agents include, for example, class I interferons (such as α-, β- and omega interferons, tau-interferons, consensus interferons and asialo-interferons), class II interferons (such as γ-interferons), pegylated interferons, levovirin and Ceplene™ (maxamine).

The term “inhibitor of HCV NS3 protease” as used herein means an agent (compound or biological) that is effective to inhibit the function of HCV NS3 protease in a mammal. Inhibitors of HCV NS3 protease include, for example, those compounds described in WO 99/07733, WO 99/07734, WO 00/09558, WO 00/09543, WO 00/59929 or WO 02/060926, and the Vertex/Eli Lilly pre-development candidate identified as VX-950 or LY-570310. Particularly, compounds#2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926, can be used in combination with the compounds of the present invention.

The term “inhibitor of another target in the HCV life cycle” means an agent (compound or biological) that is effective to inhibit the formation and/or replication of HCV in a mammal other than by inhibiting the function of HCV protease. This includes agents that interfere with either host or HCV viral mechanisms necessary for the formation and/or replication of HCV in a mammal. Inhibitors of another target in the HCV life cycle include, for example, agents that inhibit a target selected from an HCV helicase, such as an HCV RNA helicase, an HCV polymerase, such as an HCV RNA-dependent RNA polymerase, an HCV NS2-NS3 protease and an HCV IRES (Internal Ribosome Entry Site) translation. Agents that inhibit HCV polymerase include, for example, inhibitors of HCV NS5B polymerase. Inhibitors of HCV polymerase include non-nucleosides, for example, those compounds described in:

U.S. application Ser. No. 10/198,680, herein incorporated by reference in its entirety, which corresponds to PCT/CA02/01127, both filed 18 Jul. 2002 (Boehringer Ingelheim),

U.S. application Ser. No. 10/198,384, herein incorporated by reference in its entirety, which corresponds to PCT/CA02/01128, both filed 18 Jul. 2002 (Boehringer Ingelheim),

U.S. application Ser. No. 10/198,259, herein incorporated by reference in its entirety, which corresponds to PCT/CA02/01129, both filed 18 Jul. 2002 (Boehringer Ingelheim),

WO 02/100846 A1 and WO 02/100851 A2 (both Shire),

WO 01/85172 A1 and WO 02/098424 A1 (both GSK),

WO 00/06529 and WO 02/06246 A1 (both Merck),

WO 01/47883 and WO 03/000254 (both Japan Tobacco) and

EP 1 256 628 A2 (Agouron).

Furthermore other inhibitors of HCV polymerase also include nucleoside analogs, for example, those compounds described in:

WO 01/90121 A2 (Idenix),

WO 02/069903 A2 (Biocryst Pharmaceuticals Inc.), and

WO 02/057287 A2 and WO 02/057425 A2 (both Merck/Isis).

Specific examples of inhibitors of an HCV polymerase, include JTK-002, JTK-003 and JTK-109 (Japan Tobacco).

The terms “HCV replication” and “replication of HCV” mean the replication of the HCV virus as a whole or the replication of the HCV RNA genome. Thus, “HCV replication inhibitory activity” is the activity of a substance to either inhibit replication of the HCV virus or inhibit replication of the HCV RNA genome.

The term “HIV inhibitor” means an agent (compound or biological) that is effective to inhibit the formation and/or replication of HIV in a mammal. This includes agents that interfere with either host or viral mechanisms necessary for the formation and/or replication of HIV in a mammal. HIV inhibitors include, for example, nucleosidic inhibitors, non-nucleosidic inhibitors, protease inhibitors, fusion inhibitors, integrase inhibitors and entry inhibitors. Examples of HIV inhibitors include Viramune® (nevirapine) and tipranavir.

The term “HAV inhibitor” means an agent (compound or biological) that is effective to inhibit the formation and/or replication of HAV in a mammal. This includes agents that interfere with either host or viral mechanisms necessary for the formation and/or replication of HAV in a mammal. HAV inhibitors include Hepatitis A vaccines, for example, Havrix® (GlaxoSmithKline), VAQTA® (Merck) and Avaxim® (Aventis Pasteur).

The term “HBV inhibitor” means an agent (compound or biological) that is effective to inhibit the formation and/or replication of HBV in a mammal. This includes agents that interfere with either host or viral mechanisms necessary for the formation and/or replication of HBV in a mammal. HBV inhibitors include, for example, agents that inhibit HBV viral DNA polymerase or HBV vaccines. Specific examples of HBV inhibitors include emtricitabine, lamivudine (Epivir-HBV®), famciclovir, tenofovir, adefovir dipivoxil, entecavir, FTC (Coviracil®), DAPD (DXG), L-FMAU (Clevudine®), AM365 (Amrad), Ldt (telbivudine), monoval-LdC (valtorcitabine), ACH-126,443 (L-Fd4C) (Achillion), MCC478 (Eli Lilly), racivir (RCV), fluoro-L and D nucleosides, robustaflavone, ICN 2001-3 (ICN), Bam 205 (Novelos), XTL-001 (XTL), imino-sugars (Nonyl-DNJ) (Synergy), HepBzyme; and immunomodulator products such as: interferon alpha 2b, HE2000 (Hollis-Eden), theradigm (Epimmune), EHT899 (Enzo Biochem), thymosin alpha-1 (Zadaxin®), HBV DNA vaccine (PowderJect), HBV DNA vaccine (Jefferon Center), HBV antigen (OraGen), BayHep B® (Bayer), Nabi-HB® (Nabi) and anti-hepatitis B (Cangene); and HBV vaccine products such as the following: Engerix B, Recombivax HB, GenHevac B, Hepacare, Bio-Hep B, TwinRix, Comvax and Hexavac.

The term “liver immunoprotective agent” means an agent (compound or biological) that is effective to protect the liver, e.g., a newly transplanted liver, from the immune response of the host. An example of such an agent is IDN-6556 (IDUN Pharmaceuticals, Inc.).

The term “class I interferon” means an interferon selected from a group of interferons that all bind to receptor type I. This includes both naturally and synthetically produced class I interferons. Examples of class I interferons include α-, β-, omega interferons, tau-interferons, consensus interferons, asialo-interferons.

The term “class II interferon” means an interferon selected from a group of interferons that all bind to receptor type II. This includes both naturally and synthetically produced class II interferons. Examples of class II interferons include y-interferons.

The term “kit” means any packaging that contains at least a first container containing a first pharmaceutical composition and at least a second container containing a second pharmaceutical composition. In one embodiment, the first and second container are the same container, i.e., at least one container in the packaging contains both the first and second pharmaceutical compositions. The first and second pharmaceutical compositions can be in forms suitable for the same route of administration or for different routes of administration. In another embodiment, the first and second pharmaceutical compositions in the kit are each in unit dosage form.

The term “acute HCV infection” means an infection with a duration of up to six months.

The term “chronic HCV infection” means an infection with a duration of more than six months.

The term “viral load” with respect to HCV in a mammal means the number of HCV mRNA genome copies per ml of serum present in the mammal. The viral load value can be measured, for example, by mRNA-PCR quantitation in blood samples using Cobas Amplicor HCV Monitor v 2.0 (Roche Diagnostics); Amplicor HCV v 2.0, Roche Diagnostics; HCV Transcription Mediated Amplification (TMA) assay, Bayer Diagnostics; HCV RNA Qualitative Testing (TMA) assay, Bayer Diagnostics; the HCV branched DNA (bDNA) assay version 3.0, Bayer Diagnostics; Versant HCV RNA 3.0 Assay (bDNA), Bayer Diagnostics, and Superquant assay, National Genetics Institute (Los Angeles, Calif.).

The term “at least 1 log lower” with respect to the reduction of HCV viral load in a mammal means a reduction of HCV viral load to a level which is ≦0.1× the viral load of HCV in the mammal at the initiation of the treatment in accordance with the invention.

The term “at least 2 log lower” with respect to the reduction of HCV viral load in a mammal means a reduction of HCV viral load to a level which is ≦0.01× the viral load of HCV in the mammal at the initiation of the treatment in accordance with the invention.

The term “at least 3 log lower” with respect to the reduction of HCV viral load in a mammal means a reduction of HCV viral load to a level which is ≦0.001× the viral load of HCV in the mammal at the initiation of the treatment in accordance with the invention.

The term “exposed to HCV” means any physical contact with HCV. Examples of exposure include accidental entry of HCV into the blood stream, for example by syringe needle prick.

The term “infected with HCV” means the measurable presence of HCV particles in the blood.

The term “non-responsive” with respect to prior treatment for HCV means that the patient did not experience any significant HCV viral load reduction during the prior treatment or experienced a break-through during the prior treatment, so that the patient's HCV viral load is measurable at the end of the prior treatment.

The term “relapsed” with respect to a patient treated for HCV infection means that at some point in time after the conclusion of the patient's treatment the patient's HCV viral load increased from an undetectable to a measurable level.

The terms “treating” or “treatment” with respect to the treatment of a disease-state in a patient include:

(i) inhibiting or ameliorating the disease-state in a patient, e.g., arresting or slowing its development; or

(ii) relieving the disease-state in a patient, i.e., causing regression or cure of the disease-state.

The term “patient” includes human and non-human mammals.

II. Embodiments of the Invention

The present invention is based on the discovery that Compound (1) administered in an oral pharmaceutical composition at a selected dosage range was highly effective at reducing the viral load of HCV infected patients. The degree of viral load reduction upon the administration of Compound (1) was a significant and unexpected finding, with some patients even experiencing up to a 3 log reduction in viral load within 48 hours after the first administration of Compound (1). Embodiments of the present invention therefore include various oral pharmaceutical compositions, kits and methods of treating and preventing Hepatitis C Viral (HCV) infections wherein Compound (1), or a pharmaceutically acceptable salt thereof, is used in a selected dosage range.

II.A. Oral Pharmaceutical Compositions

In a general embodiment, the oral pharmaceutical composition according to the present invention comprises about 25 mg to 500 mg of Compound (1), or a pharmaceutically acceptable salt thereof, dissolved in at least one solvent selected from polyethylene glycol, ethanol, propylene glycol and water, or mixtures thereof, preferably a mixture of polyethylene glycol and ethanol, and optionally further comprising a suitable antioxidant, for example, sodium sulfite, Vitamin E TPGF, propyl gallate or ascorbic acid. The antioxidant is preferably ascorbic acid. More specific embodiments of this composition are wherein the Compound (1) is present in an amount of about 25 mg to 150 mg, or about 150 mg to 250 mg, or about 250 mg to 500 mg. Specific dosage levels include about 25 mg, about 200 mg and about 500 mg, preferably about 200 mg.

The relative amounts of the solvents and antioxidant can be easily adjusted and optimized by a person skilled in the art depending on particular composition to be used so that optimum results are achieved. In a more specific embodiment, however, the weight ratio of polyethylene glycol to ethanol in this preferred solvent mixture is in the range of 75:25 to 95:5 (w/w), preferably 80:20 (w/w). A preferred polyethylene glycol is Polyethylene Glycol 400 (PEG 400) but other polyethylene glycols can be used depending on the particular composition and other ingredients. The amount of antioxidant is preferably about 0.1%. More specific embodiments of this composition are wherein the Compound (1) is present in an amount of about 25 mg to 150 mg, or about 150 mg to 250 mg, or about 250 mg to 500 mg. Specific dosage levels include about 25 mg, about 200 mg and about 500 mg, preferably about 200 mg.

In another specific embodiment, the oral pharmaceutical composition comprises about 200 mg of Compound (1) dissolved in a solvent mixture of Polyethylene Glycol 400/ethanol (75:25 to 95:5, w/w, preferably 80:20 w/w), optionally containing 0.1% ascorbic acid.

The oral pharmaceutical compositions of the invention may contain one or more additional active agents selected, for example, from antiviral agents, immunomodulatory agents, other inhibitors of HCV NS3 protease, inhibitors of another target in the HCV life cycle, HIV inhibitors, HAV inhibitors, HBV inhibitors and liver immunoprotective agents. Examples of such agents are provided in the Definitions section above. Specific preferred examples of some of these agents are listed below:

(1) antiviral agents: ribavirin and amantadine.

(2) immunomodulatory agents: class I interferons, class II interferons and pegylated interferons.

(3) inhibitor of another target in the HCV life cycle that inhibits a target selected from: an HCV helicase, an HCV polymerase, an HCV IRES translation and an HCV NS2-NS3 protease.

(4) HIV inhibitors: nucleosidic inhibitors, non-nucleosidic inhibitors, protease inhibitors, fusion inhibitors, integrase inhibitors and entry inhibitors.

(5) HBV inhibitors: agents that inhibit HBV viral DNA polymerase or is an HBV vaccine.

More specific embodiments include the following specific combination compositions:

(a) An oral pharmaceutical composition comprising about 25 mg to 500 mg of Compound (1), ribavirin and an α-interferon.

(b) An oral pharmaceutical composition comprising about 25 mg to 500 mg of Compound (1), ribavirin and pegylated α-interferon.

The pharmaceutical compositions in this embodiment of the invention are administered orally as a liquid for the treatment or prevention of HCV in a mammal. These pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The pharmaceutical compositions of this invention may be orally administered as a solution. If desired, certain sweetening and/or flavoring and/or coloring agents may be added. Other suitable vehicles or carriers for the above noted compositions can be found in standard pharmaceutical texts, e.g. in “Remington's Pharmaceutical Sciences”, 19^{th} ed., Mack Publishing Company, Easton, Pa., 1995.

Typically, the pharmaceutical compositions of this invention will be administered twice daily (bid) so as to provide a daily dosage of about 50 mg to 1000 mg of Compound (1), or a pharmaceutically acceptable salt thereof, to the patient, which daily dosage was found to be highly effective at reducing the HCV viral load of HCV infected patients. Such administration can be used for chronic or acute HCV therapy, and for the treatment of various patient population groups as described hereinafter. The relative amounts of active ingredients that may be combined with the solvents to produce a single dosage form will vary. A typical preparation will contain from about 5% to about 95% active compound (w/w). Preferably, such preparations contain from about 20% to about 80% active compound.

When the compositions of this invention comprise a combination of Compound (1), or a pharmaceutically acceptable salt thereof, and one or more additional therapeutic or prophylactic agents as described above, both the compound and the additional agent(s) should be present at dosage levels of between about 10 to 100%, and more preferably between about 10 and 80% of the dosage normally administered in a monotherapy regimen.

II.B Kits

A general embodiment is directed to a kit comprising:

(a) about 25 mg to 500 mg of Compound (1), or a pharmaceutically acceptable salt thereof; and

(b) at least one of the following additional agents: an antiviral agent, an immunomodulatory agent, another inhibitor of HCV NS3 protease, an inhibitor of another target in the HCV life cycle, an HIV inhibitor, an HAV inhibitor, an HBV inhibitor or a liver immunoprotective agent.

The Compound (1), or pharmaceutically acceptable salt thereof, is generally present in the form of a first pharmaceutical composition in the kit, and the additional agent is generally present in the form of a second pharmaceutical composition in the kit, with additional pharmaceutical compositions for any additional agents. The first, second, etc, pharmaceutical compositions in the kit can each be in separate containers within the kit or can be in the same container in the kit. The pharmaceutical compositions in the kit can be in forms suitable for the same route of administration or for different routes of administration.

In another embodiment, the pharmaceutical compositions in the kit are each in unit dosage form. Any conventional dosage forms can be used for the pharmaceutical compositions in the kit, e.g., tablets, capsules (e.g., hard or soft gelatin capsules), aqueous suspensions and solutions, or sterile injectable preparations such as sterile injectable aqueous or oleaginous suspensions, and these pharmaceutical compositions can be administered to the patient in a conventional manner consistent with the dosage form. Examples of soft gelatin capsules that can be used include those disclosed in EP 649651 B1 and U.S. Pat. No. 5,985,321.

The pharmaceutical compositions in the kit may contain any conventional non-toxic pharmaceutically-acceptable carriers, adjuvants or vehicles. In some cases, the pH of the formulations may be adjusted with pharmaceutically acceptable acids, bases or buffers to enhance the stability of the formulated compound or its delivery form. If desired, certain sweetening and/or flavoring and/or coloring agents may be added. Other suitable vehicles or carriers for the above noted compositions can be found in standard pharmaceutical texts, e.g. in “Remington's Pharmaceutical Sciences”, 19^{th} ed., Mack Publishing Company, Easton, Pa., 1995.

The kits according to the invention can be used for combination therapy of HCV wherein at least two of the therapeutic agents are in separate pharmaceutical compositions. When the patient is coinfected with HCV and HIV, at least one of the pharmaceutical compositions preferably contains at least one HIV inhibitor. When the patient is coinfected with HCV and HAV, at least one of the pharmaceutical compositions preferably contains at least one HAV inhibitor. When the patient is coinfected with HCV and HBV, at least one of the pharmaceutical compositions preferably contains at least one HBV inhibitor.

II.C Methods of Treating and Preventing HCV

In another embodiment, the present invention is directed to a method of treating or preventing HCV infection in a mammal comprising administering to said mammal about 50 mg to 1000 mg of Compound (1), or a pharmaceutically acceptable salt thereof, per day in single or multiple doses. Other more specific dosage ranges include about 50 mg to 300 mg, or about 300 mg to 500 mg, or about 500 mg to 1000 mg of Compound (1) or a pharmaceutically acceptable salt thereof, per day. Specific daily dosage levels include about 50 mg, about 400 mg and about 1000 mg, preferably about 400 mg.

Compound (1), or a pharmaceutically acceptable salt thereof, at a selected dosage level is typically administered to the patient via a pharmaceutical composition. The pharmaceutical composition may be administered orally, parenterally or via an implanted reservoir. Oral administration or administration by injection are preferred. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intra-articular, intrasynovial, intrasternal, intrathecal, and intralesional injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example. Tween 80) and suspending agents.

In one further embodiment, Compound (1), or a pharmaceutically acceptable salt thereof, is administered by an oral pharmaceutical composition comprising Compound (1), or a pharmaceutically acceptable salt thereof, at a selected dosage level as described above, and at least one pharmaceutically acceptable carrier or diluent. The oral pharmaceutical compositions may be orally administered in any orally acceptable dosage form including, but not limited to, tablets, capsules (e.g., hard or soft gelatin capsules), and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. Examples of soft gelatin capsules that can be used include those disclosed in EP 649651 B1 and U.S. Pat. No. 5,985,321. When aqueous suspensions are administered orally, the active ingredient is combined with emulsifying and suspending agents. If desired, certain sweetening and/or flavoring and/or coloring agents may be added.

Other suitable vehicles or carriers for the above noted formulations and compositions can be found in standard pharmaceutical texts, e.g. in “Remington's Pharmaceutical Sciences”, 19^{th} ed., Mack Publishing Company, Easton, Pa., 1995.

In a specific embodiment, the oral pharmaceutical composition comprises Compound (1), or a pharmaceutically acceptable salt thereof, in a selected dosage level above, dissolved in at least one solvent selected from polyethylene glycol, ethanol, propylene glycol, water or mixtures thereof, preferably a mixture of polyethylene glycol and ethanol, and optionally further comprising a suitable antioxidant, for example, sodium sulfite, Vitamin E TPGF, propyl gallate or ascorbic acid. The antioxidant is preferably ascorbic acid.

In another embodiment, the pharmaceutical composition that is administered further comprises at least one agent selected from: an antiviral agent, an immunomodulatory agent, another inhibitor of HCV NS3 protease, an inhibitor of another target in the HCV life cycle, an HIV inhibitor, an HAV inhibitor, an HBV inhibitor and a liver immunoprotective agent. Examples of such agents are provided in the Definitions section above. Specific preferred examples of some of these agents are listed below:

(1) antiviral agents: ribavirin and amantadine.

(2) immunomodulatory agents: class I interferons, class II interferons and pegylated interferons.

(3) inhibitor of another target in the HCV life cycle that inhibits a target selected from: an HCV helicase, an HCV polymerase, an HCV IRES translation and an HCV NS2-NS3 protease.

(4) HIV inhibitors: nucleosidic inhibitors, non-nucleosidic inhibitors, protease inhibitors, fusion inhibitors, integrase inhibitors and entry inhibitors.

(5) HBV inhibitors: agents that inhibit HBV viral DNA polymerase or is an HBV vaccine.

The methods of the present invention can be used to treat or prevent HCV infection in a variety of patient groups, for example:

I. Patients having HCV of the genotype 1 variety;

II. Patients having HCV of the non-genotype 1 variety;

III. Patients having acute HCV infection;

IV. Patients having chronic HCV infection;

V. Patients wherein the viral load of HCV in the patient when Compound (1) or a pharmaceutically acceptable salt thereof, is first administered to said patient is less than 2 million copies per ml of blood plasma.

VI. Patients wherein the viral load of HCV in the patient when Compound (1) or a pharmaceutically acceptable salt thereof is first administered to said patient is equal to or greater than 2 million copies per ml of blood plasma.

VII. Patients having no liver fibrosis.

VIII. Patients having mild, moderate or severe liver fibrosis, or cirrhosis.

IX. Patients that are human, including various races, e.g. african-american, Caucasian, asian, etc., adults, children, male and female.

X. Patients infected with HCV at the time when Compound (1) or a pharmaceutically acceptable salt thereof is first administered.

XI. Patients exposed to but not infected with HCV at the time when Compound (1) or a pharmaceutically acceptable salt thereof is first administered

XII. Patients that have not been exposed to HCV at the time when Compound (1) or a pharmaceutically acceptable salt thereof is first administered.

XIII. Patients infected with HCV but no other virus at the time when Compound (1) or a pharmaceutically acceptable salt thereof is first administered.

XIV. Patients coinfected with HCV and HIV when Compound (1) or a pharmaceutically acceptable salt thereof is first administered to said patient.

XV. Patients coinfected with HCV and HAV when Compound (1) or a pharmaceutically acceptable salt thereof is first administered to said patient.

XVI. Patients coinfected with HCV and HBV when Compound (1) or a pharmaceutically acceptable salt thereof, is first administered to said patient.

XVII. Patients that have never before been treated for HCV infection.

XVIII. Patients that have been previously treated for HCV infection but were either non-responsive to said prior treatment or relapsed after the conclusion of said prior treatment.

Of course, the treated patient will generally fall within a number of the above-listed patient groups simultaneously. For example, patients having the following combination of characteristics are contemplated as examples of patient types that may be treated:

(1) Patients having chronic HCV infection of the genotype 1 variety and having no liver fibrosis;

(2) Patients having chronic HCV infection of the genotype 1 variety and having mild or moderate liver fibrosis;

(3) Patients having chronic HCV infection of the genotype 1 variety and having severe liver fibrosis or cirrhosis;

(4) Patients having acute HCV infection of the genotype 1 variety and having no liver fibrosis;

(5) Patients having acute HCV infection of the genotype 1 variety and having mild or moderate liver fibrosis;

(6) Patients having acute HCV infection of the genotype 1 variety and having severe liver fibrosis or cirrhosis;

Patients having other combinations of patient group characteristics from the above list I-XVIII are of course contemplated within the scope of the present invention.

With respect to patients coinfected with HCV and HIV (Patient Class XIV), the method may further comprise additionally administering to said patient at least one HIV inhibitor in an amount effective to treat HIV infection in said patient. Examples of HIV inhibitors are set forth in the Definitions section above. Specific preferred examples are: nucleosidic inhibitors, non-nucleosidic inhibitors, protease inhibitors, fusion inhibitors, integrase inhibitors and entry inhibitors, or combinations thereof (e.g. HAART).

With respect to patients coinfected with HCV and HAV (Patient Class XV), the method may further comprise additionally administering to said patient at least one HAV inhibitor in an amount effective to treat HAV infection in said patient. Examples of HAV inhibitors are set forth in the Definitions section above.

With respect to patients coinfected with HCV and HBV (Patient Class XVI), the method may further comprise additionally administering to said patient at least one HBV inhibitor in an amount effective to treat HBV infection in said patient. Examples of HBV inhibitors are set forth in the Definitions section above. Specific preferred examples are: an agent that inhibits HBV viral DNA polymerase or is an HBV vaccine.

The methods of the present invention can be used to achieve various levels of reduction in HCV viral load in a patient, in some cases up to a 3 log reduction, depending on factors such as the particular patient's condition and the dosage level of Compound (1) or pharmaceutically acceptable salt thereof that is used in the treatment.

Preferred embodiments based on viral load reduction levels are as follows:

I. Wherein at 48 hours after the first administration of Compound (1) or a pharmaceutically acceptable salt thereof to the patient, the viral load of HCV in the patient is at least 1 log lower than the viral load of HCV in the patient when Compound (1) is first administered to said patient.

II. Wherein at 48 hours after the first administration of Compound (1) or a pharmaceutically acceptable salt thereof to the patient, the viral load of HCV in the patient is at least 2 log lower than the viral load of HCV in the patient when Compound (1) is first administered to said patient.

III. Wherein at 48 hours after the first administration of Compound (1) or a pharmaceutically acceptable salt thereof to the patient, the viral load of HCV in the patient is at least 3 log lower than the viral load of HCV in the patient when Compound (1) is first administered to said patient.

Specific optimal dosage and treatment regimens for any particular patient will of course depend upon a variety of factors, including the age, body weight, general health status, sex, diet, time of administration, rate of excretion, drug combination, the severity and course of the infection, the patient's disposition to the infection and the judgment of the treating physician. Generally, treatment is initiated with small dosages substantially less than the optimum dose. Thereafter, the dosage is increased by small increments until the optimum effect under the circumstances is reached. In general, the compound is most desirably administered at a concentration level that will generally afford antivirally effective results without causing any harmful or deleterious side effects.

Additional specific embodiments of the method of the present invention include the following:

I. A method wherein said patient is a human, wherein about 50 to 1000 mg, for example about 300 to 500 mg, for example about 400 mg, of Compound (1) is administered to said human per day, wherein the HCV in the human is of the genotype 1 variety, wherein the HCV infection in the human is chronic HCV infection, and wherein at 48 hours after the first administration of Compound (1) to the human, the viral load of HCV in the human is at least 1 log lower than the viral load of HCV in the human when Compound (1) is first administered to said human.

II. A method as in I above, wherein at 48 hours after the first administration of Compound (1) to the human, the viral load of HCV in the human is at least 2 log lower than the viral load of HCV in the human when Compound (1) is first administered to said human.

III. A method as in I above, wherein at 48 hours after the first administration of Compound (1) to the human, the viral load of HCV in the human is at least 3 log lower than the viral load of HCV in the human when Compound (1) is first administered to said human.

Combination therapy is contemplated wherein Compound (1), or a pharmaceutically acceptable salt thereof, is co-administered with at least one additional agent selected from: an antiviral agent, an immunomodulatory agent, another inhibitor of HCV NS3 protease, an inhibitor of another target in the HCV life cycle, an HIV inhibitor, an HAV inhibitor an HBV inhibitor, and a liver immunoprotective agent. Examples of such agents are provided in the Definitions section above. These additional agents may be combined with Compound (1), or a pharmaceutically acceptable salt thereof, to create a single pharmaceutical dosage form. Alternatively these additional agents may be separately administered to the patient as part of a multiple dosage form, for example, using a kit as described above. Such additional agents may be administered to the patient prior to, concurrently with, or following the administration of Compound (1), or a pharmaceutically acceptable salt thereof.

II.D Use of Compound (1) for HCV Assay Validation and HCV Assay Control

In another embodiment, the present invention is directed to a method for validating an assay useful for determining whether one or more substances, alone or in combination, inhibit(s) the replication of HCV, comprising: a) running a control substance in said assay, wherein the control substance comprises the compound of formula (1) or a pharmaceutically acceptable salt thereof, and b) determining the HCV replication inhibitory activity of said control substance in the assay. In this embodiment, the compound of formula (1) or a pharmaceutically acceptable salt thereof, is used to determine whether the assay under evaluation can be used to measure HCV replication inhibitory activity. Optionally, an additional step in this validation process may comprise comparing said HCV replication inhibitory activity of said control substance as determined in step (b) to the HCV replication inhibitory activity of said control substance when measured in a different assay. This further step is useful, for example, to evaluate the accuracy of assay under evaluation in determining the HCV replication inhibitory activity of a substance by comparison to the result obtained for that substance in a known standard assay.

Another embodiment is directed to a method for determining the relative effectiveness of one or more substances, alone or in combination, to inhibit the replication of HCV, comprising: a) running said substance(s) in an assay that is useful for determining whether a substance inhibits the replication of HCV; b) determining the HCV replication inhibitory activity of said substance(s) in said assay; and c) comparing said HCV replication inhibitory activity to the HCV replication inhibitory activity of a control substance that is determined in an identical or different assay, wherein the control substance comprises a compound of formula (1) or a pharmaceutically acceptable salt thereof. In this method, the a compound of formula (1) or a pharmaceutically acceptable salt thereof, is used as a control substance in order to determine the relative effectiveness of a test substance(s) to inhibit the replication of HCV. That is, the assay results are compared to determine whether the test substance(s) is more, less or equally as effective as the compound of formula (1), or a pharmaceutically acceptable salt thereof, in the inhibition of HCV replication.

In both the HCV assay validation and HCV assay control embodiments discussed above, the assays used to determine HCV replication inhibitory activity can be an in-vitro assay, e.g., a cell-based assay, or an in-vivo assay, e.g., an animal-based assay. Testing in humans is one type of animal-based assay that is contemplated.

In order that this invention be more fully understood, the following examples are set forth. These examples are for the purpose of illustrating embodiments of this invention, and are not to be construed as limiting the scope of the invention in any way.

### III. EXAMPLES

A. Pharmaceutical Compositions

Several compositions may be prepared in the form of oral solutions of Compound (1) powder dissolved in a mixed solvent of PEG 400:Ethanol=80:20 w/w, as follows:

Solvent 1

| | |
|---|---|
| Substance: | PEG 400 |
| Volume: | 14 ml per vial |

Solvent 2

| | |
|---|---|
| Substance: | Ethanol absolute (absolute [PhEur III/USP XXIV]) |
| Volume: | 5 ml added to Solvent 1 |

Reconstitution Bulk Solvent

Substance: PEG 400:Ethanol 80%-20% mixture (w/w, vehicle)

Volume: 19 ml (14 ml PEG 400+5 ml Ethanol absolute)

Volume for reconstitution: 5 ml per administration vial

Bulk Solvent Preparation (PEG 400: Ethanol=80:20, W/W):

Attach a needle to a 5 mL syringe and draw up 5 mL of ethanol; place tip of needle to inside wall of vial containing 14 mL PEG 400 and gently expel 5 mL of ethanol into vial; Cap the vial immediately to avoid ethanol evaporation; Shake the vial for no less than 15 seconds; Set vial aside for 5 minutes before use or until there are no air bubbles present in solvent.

Solution Preparation Instructions:

1. Remove the Teflon cap from the vial containing the solvent and replace it with the Adapta-cap (blue).

2. Screw the Adapta-cap on the vial containing the solvent. Do not overtighten by force. Unscrew the Adapta-cap by a ¼ turn to avoid a vacuum.

3. With the 5 ml Baxa oral administration syringe, draw up 5 mL of the bulk solvent. Do not push solvent back into the vial.

4. Obtain a vial containing an amount of Compound (1) powder appropriate for the selected individual strength.

Note: Compound (1) powder has a high tendency to adhere to the sides of the vial and caps. While keeping the vial capped, tap the vial several times on a hard surface to get the powder down into the vial to minimize any potential loss. When removing the cap, remove slowly and set with teflon-side up to minimize any potential loss of drug that may be adhered to the interior of the cap.

5. Carefully remove the Teflon cap from the vial containing Compound (1) powder.

6. Carefully and slowly introduce the bulk solvent into the powder vial by placing the tip of the syringe against the side of the vial and injecting down the sides of the vial (do not inject directly into the vial since the powder could be expelled from the vial).

7. Carefully reapply the Teflon cap.

8. Cover the vial with aluminum foil.

Note: The solution is light sensitive. Keep away from direct sunlight and cover with aluminium foil whenever possible.

9. Vigorously shake vial by hand for 10 minutes. Allow the powder to sit for 10 minutes. Vigorously shake the vial again for 3 minutes. Allow the vial to sit for 2 minutes. If any particles remain, vigorously shake by hand again for 3 minutes or until all of the powder is dissolved.

10. Let the vial stand upright for approximately 2 minutes to allow the solution to drain down the sides.

### Examples of Oral Dose Solutions

The following are examples of oral dose solutions prepared in the manner described above:

Solution 1:

| | |
|---|---|
| Unit strength: | 25 mg/3 ml |
| Concentration: | 8.33 mg/ml |
| Amount per vial: | 41.7 ml |
| Daily dose: | 50 mg in 6 ml |

Solution 2:

| | |
|---|---|
| Unit strength: | 200 mg/3 ml |
| Concentration: | 66.7 mg/ml |
| Amount per vial: | 333.3 ml |
| Daily dose: | 400 mg in 6 ml |

Solution 3:

| | |
|---|---|
| Unit strength: | 500 mg/3 ml |
| Concentration: | 166.7 mg/ml |
| Amount per vial: | 833.3 ml |
| Daily dose: | 1000 mg in 6 ml |

B. Administration of Oral Dose Solutions

A method for administering the oral solutions is described below:

1. Carefully remove the Teflon cap from the vial containing the reconstituted solution and replace it with the Adapta-cap (blue).

2. Screw the Adapta-cap on the vial containing the reconstituted solution. Do not overtighten by force. Unscrew the Adapta-cap by a ¼ turn to avoid a vacuum.

3. Attach a 5 mL Baxa oral administration syringe to the port on the Adapta-cap.

4. Hold the capped vial inverted for 1-2 minutes to allow for complete drainage of the solution.

5. Withdraw 3 mL of the reconstituted solution and place the blue tip cap on the end of the syringe to prevent leakage.

6. Once prepared, the dose can be used immediately or it may be stored up to 3 hours, at room temperature, provided the syringe is completely wrapped in aluminum foil to protect it from light.

7. Remove the tip cap and administer the dose to the patient orally.

We claim:

1) An oral pharmaceutical composition comprising about 25 mg to 500 mg of the following Compound (1) or a pharmaceutically acceptable salt thereof: 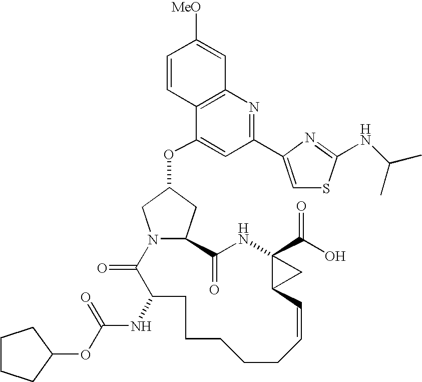
dissolved in at least one solvent selected from polyethylene glycol, ethanol, propylene glycol and water, and mixtures thereof.

2) An oral pharmaceutical composition according to claim 1, wherein said composition comprises about 25 mg to 500 mg of Compound (1) or a pharmaceutically acceptable salt thereof, dissolved in a mixture of a polyethylene glycol and ethanol.

3) A kit comprising:
(a) about 25 mg to 500 mg of Compound (1), or a pharmaceutically acceptable salt thereof; and
(b) at least one of the following additional agents: an antiviral agent, an immunomodulatory agent, another inhibitor of HCV NS3 protease, an inhibitor of another target in the HCV life cycle, an HIV inhibitor, an HAV inhibitor, an HBV inhibitor or a liver immunoprotective agent.

4) A method of treating or preventing HCV infection in a mammal comprising administering to said mammal about 50 mg to 1000 mg of the following Compound (1), or a pharmaceutically acceptable salt thereof, per day in single or multiple doses:

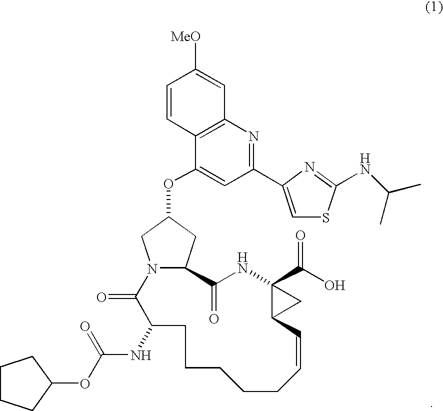

5) A method according to claim 4, wherein about 50 mg to 300 mg of Compound (1) or a pharmaceutically acceptable salt thereof, is administered to said mammal per day.

6) A method according to claim 4, wherein about 300 mg to 500 mg of Compound (1) or a pharmaceutically acceptable salt thereof, is administered to said mammal per day.

7) A method according to claim 4, wherein about 500 to 1000 mg of Compound (1) or a pharmaceutically acceptable salt thereof, is administered to said mammal per day.

8) A method according to claim 4, wherein the HCV is of the genotype 1 variety.

9) A method according to claim 4, wherein the HCV is chronic HCV of the genotype 1 variety.

10) A method according to claim 4, wherein at 48 hours after the first administration of Compound (1) or a pharmaceutically acceptable salt thereof to the mammal, the viral load of HCV in the mammal is at least 1 log lower than the viral load of HCV in the mammal when Compound (1) is first administered to said mammal.

11) A method according to claim 4, wherein at 48 hours after the first administration of Compound (1) or a pharmaceutically acceptable salt thereof, to the mammal, the viral load of HCV in the mammal is at least 2 log lower than the viral load of HCV in the mammal when Compound (1) is first administered to said mammal.

12) A method according to claim 4, wherein at 48 hours after the first administration of Compound (1) or a pharmaceutically acceptable salt thereof, to the mammal, the viral load of HCV in the mammal is at least 3 log lower than the viral load of HCV in the mammal when Compound (1) is first administered to said mammal.

13) A method according to claim 4, wherein said mammal is a human, wherein about 50 to 1000 mg of Compound (1) is administered to said human per day, wherein the HCV infection is chronic HCV infection of the genotype 1 variety, and wherein at 48 hours after the first administration of Compound (1) to the mammal, the viral load of HCV in the mammal is at least 1 log lower than the viral load of HCV in the mammal when Compound (1) is first administered to said mammal.

14) A method according to claim 13, wherein at 48 hours after the first administration of Compound (1) to the mammal, the viral load of HCV in the mammal is at least 2 log lower than the viral load of HCV in the mammal when Compound (1) is first administered to said mammal.

15) A method according to claim 13, wherein at 48 hours after the first administration of Compound (1) to the mammal, the viral load of HCV in the mammal is at least 3 log lower than the viral load of HCV in the mammal when Compound (1) is first administered to said mammal.

16) A method according to claim 13, wherein about 300 to 500 mg of Compound (1) is administered to said human per day.

17) A method according to claim 14, wherein about 300 to 500 mg of Compound (1) is administered to said human per day.

18) A method according to claim 15, wherein about 300 to 500 mg of Compound (1) is administered to said human per day.

19) A method according to claim 13, wherein about 400 mg of Compound (1) is administered to said human per day.

20) A method according to claim 14, wherein about 400 mg of Compound (1) is administered to said human per day.

21) A method according to claim 15, wherein about 400 mg of Compound (1) is administered to said human per day.

22) A method according to claim 4, wherein Compound (1), or a pharmaceutically acceptable salt thereof, is administered by an oral pharmaceutical composition comprising Compound (1), or a pharmaceutically acceptable salt thereof, and at least one pharmaceutically acceptable carrier or diluent.

23) A method according to claim 22, wherein Compound (1) or a pharmaceutically acceptable salt thereof is administered by an oral pharmaceutical composition comprising Compound (1), or a pharmaceutically acceptable salt thereof, dissolved in at least one solvent selected from polyethylene glycol, ethanol, propylene glycol, water and mixtures thereof.

24) A method according to claim 23, wherein the solvent is a mixture of polyethylene glycol and ethanol.

25) A method according to claim 22, wherein said composition further comprises at least one agent selected from: an antiviral agent, an immunomodulatory agent, another inhibitor of an HCV NS3 protease, an inhibitor of another target in the HCV life cycle, an HIV inhibitor, an HAV inhibitor, an HBV inhibitor and a liver immunoprotective agent.

26) A method according to claim 4, wherein the Compound (1), or a pharmaceutically acceptable salt thereof, is co-administered with a least one additional agent selected from: an antiviral agent, an immunomodulatory agent, another inhibitor of an HCV NS3 protease, an inhibitor of another target in the HCV life cycle, an HIV inhibitor, an HAV inhibitor, an HBV inhibitor and a liver immunoprotective agent, and said additional agent is administered to the patient prior to, concurrently with, or following the administration of Compound (1), or a pharmaceutically acceptable salt thereof.

27) A method for validating an assay useful for determining whether one or more substances, alone or in combination, inhibit(s) the replication of HCV, comprising: a) running a control substance in said assay, wherein the control substance comprises the following Compound (1), or a pharmaceutically acceptable salt thereof, and b) determining the HCV replication inhibitory activity of said control substance in the assay:

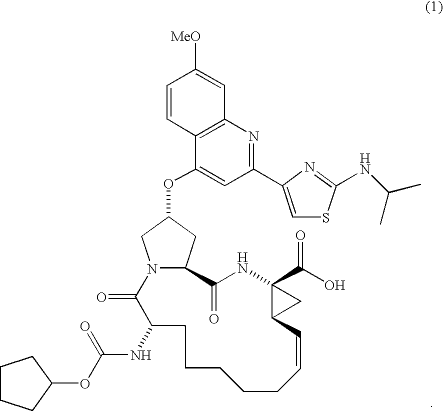

28) A method for determining the relative effectiveness of one or more substances, alone or in combination, to inhibit the replication of HCV, comprising: a) running said substance(s) in an assay that is useful for determining whether a substance inhibits the replication of HCV; b) determining the HCV replication inhibitory activity of said substance(s) in said assay; and c) comparing said HCV replication inhibitory activity to the HCV replication inhibitory activity of a control substance that is determined in an identical or different assay, wherein the control substance comprises the following Compound (1) or a pharmaceutically acceptable salt thereof: [chemical structure]
